(19) United States
(12) Patent Application Publication
(10) Pub. No.: US 2017/0145360 A1
(43) Pub. Date: May 25, 2017

(54) METHOD OF PREPARING WHITE LIQUOR FLAVORED ENHANCING PEPTIDE

(71) Applicant: South China University of Technology, Tianhe District Guangzhou,Guangdong (CN)

(72) Inventors: Mouming ZHAO, Guangzhou (CN); Guowan SU, Guangzhou (CN); Qiangzhong ZHAO, Guangzhou (CN); Rongzhong ZHAO, Guangzhou (CN); Lianzhu LIN, Guangzhou (CN)

(21) Appl. No.: 15/306,343

(22) PCT Filed: Nov. 28, 2014
(86) PCT No.: PCT/CN2014/092542

§ 371 Date: Oct. 24, 2016

(30) Foreign Application Priority Data
Jan. 6, 2014 (CN) 201410005962.6

(51) Int. Cl. C12G 3/06; C12P 21/06

(52) U.S. Cl. CPC C12G 3/06; C12P 21/06; C12G 2200/21

(57) ABSTRACT

The present invention discloses a preparation method of liquor flavor-enhancing peptide, comprising the following steps: defatted soybean meal and chloroform/ethanol are mixed uniformly; After treated by colloid mill and centrifuge, the obtained precipitate is the defatted soybean meal removal of the alcohol-soluble components; and then 55% (v/v) of ethanol aqueous solution and protease are added therein; the supernatant is condensed to obtain liquor flavor-enhancing peptide. The present invention uses 55% of ethanol solution to hydrolyze the defatted soybean meal, from one side, to precipitate and remove the macromolecule protein or polypeptides of the defatted soybean meal hydrolysate, so as to avoid precipitation and layering when added in liquors; from the other side, during hydrolysis, that ethanol can absorb abundant abnormal taste, which is easily removed through the following condensation procedure, providing guarantee for preparing high quality of flavoring peptide.

### TECHNICAL FIELD

The present invention relates to a bioactive peptide, specifically to a preparation method of liquor flavor-enhancing peptide.

### BACKGROUND ART

Liquor is a traditional distillate spirit for a long history in China, also our outstanding and precious national heritage, which stands side by side, as six notable distillate spirits in the world, with Brandy, Whisky, Vodka, Rum and Gin. Due to the difference of raw materials and preparation process, liquor with abundant special tastes is prepared. It is classified into “four basic flavor types and six subtle flavor types” according to the characters of tastes, for which the four basic flavor types are Maotai-flavor, Luzhou-flavor, Fen-flavor, Sanhua-flavor, and the six subtle flavor types are Mixed-flavor, Sesame-flavor, Feng-flavor, Te-flavor, Soybean-flavor and Dong-flavor (Medicinal-flavor). In 2004, a standard of Laobaigan-flavor was proposed. Therefore, now there form 11 flavor types for Chinese liquor.

The main components of liquor are ethanol and water, accounting for almost 98% of the total volume, however, the skeleton components and aroma materials which form the special flavor of liquor are microconstituents only about 2% of the total volume, of which the organic compounds with trace quantity are called as aroma substance or flavoring component of liquor. Till now, there have detected through modern analytical techniques more than 300 kinds of flavoring components in liquor, which are mainly organic acids, alcohols, aldehydes, ketones and esters. Since all traditional liquor are produced by fermentation, and there are lots of influencing factors during production, the quality of liquor produced from different batches or different fermentation vessels for same batch is different. Accordingly, blending is needed to unify the quality and standard before releasing to market. In addition, for all newly fermented liquor with stimulating flavor, blending and flavoring can perfect the quality of liquor and cater to the customers.

The principle of blending and flavoring is to adjust the ratio and content among the flavoring components with microcontent in liquor, in order to balance the aroma elements as much as possible and remove unpleasant flavor to make the liquor optimal. Currently, the feed stock of blending and flavoring is mainly distillate spirit of different batches or produced many years ago, as well as some flavoring precursors of microcontent.

Peptide is a polymer formed by amino acids linked through amide linkage, except for multiple physiological activities such as immunomodulatory, antioxidant and antihypertensive activities, playing important role in sensory taste in foods, which is one of the important taste components in foods. Peptide has complex function of taste, not only providing basic tastes of sour, sweet, bitter, salty and umami, but also involving and influencing formation of food flavoring, so as to increase food taste and to improve food quality, allowing the overall food taste harmonious, delicate or rich mellow, which is one of the main stock blends for high class of combined flavoring and spices.

With improvement of living standard, people pay more attention to daily diets and healthcare, therefore people introduce peptide with many physiological activities into liquors: Qu Chunsong disclosed a brewing method for a biopolypeptide Yuye Liquor, which mainly related to mix the nature liquor with biopolypeptide and then to ferment; Zou Yuandong utilized the Botrytis cinerea and yeast in microorganism to ferment the glutinous rice to produce a protein liquor abundant with micromolecule peptide and amino acids; Yang Xijuan produced polypeptide liquor by directly fermenting broad bean hydrolysate, combined with debitterizing by β-Cyclodextrin, so that the produced broad bean polypeptide liquor exhibited a umami and unadulterated taste; and Xiao Wenhe produced a freshwater protein polypeptide liquor after filtration and ageing the mixture of base liquor and freshwater fish protein polypeptide. Although these liquors all contain protein polypeptide, these only emphasize the functionality, without any reference to flavoring effect.

### CONTENT OF THE INVENTION

The chief object of the present invention is to provide a preparation method of liquor flavor-enhancing peptide, for which method defatted soybean meal is used as raw material, ethanol aqueous solution as medium, to prepare bioactive peptide by controlling enzymolysis, that peptide product is used in flavoring liquor so as to allow the liquor flavor more harmonious and the mouthfeel more unadulterated.

Another object of the present invention is to provide liquor flavor-enhancing peptide produced by the above-mentioned method.

A further object of the present invention is to provide the uses of the above-mentioned liquor flavor-enhancing peptide in liquor flavoring.

The objects of the present invention are realized by the following technical solutions:

A preparation method of liquor flavor-enhancing peptide, comprising the following steps:

(1) to remove the alcohol-soluble components from defatted soybean meal: one piece by weight of defatted soybean meal and 3-5 pieces by weight of chloroform/ethanol are mixed uniformly; after treated by colloid mill and centrifuge, the precipitate is collected after washed by water for multiple times; and the final precipitate is the defatted soybean meal removal of the alcohol-soluble components;

(2) to control enzymolysis of the defatted soybean meal: 55% (v/v) of ethanol aqueous solution is added into the defatted soybean meal prepared in step (1) until the protein concentration of the defatted soybean meal reaching to 7%-10% (w/w) of the mixture; then the protease is added therein; the enzymolysis reaction is carried at 50° C.-60° C. for 12-18 h, keeping stable ethanol concentration (55% (v/v)), after inactivated and centrifugated; the obtained supernatant is the soybean protein hydrolysate riched in flavor peptide; and then the same is condensed to obtain liquor flavor-enhancing peptide; using the mass of defatted soybean meal as calculation reference, the addition amount of protease accounts for 0.5%-1.2%; and said mixture means the system composed of the defatted soybean meal produced in step (1) and ethanol aqueous solution of 55% (v/v);

Said centrifugation is 15 min at 8000×g;

In said chloroform/ethanol mixed liquor in step (1), the ratio by volume of chloroform to ethanol is 1:(1-3);

Said protease in step (2) is protease having high tolerance to ethanol, preferably Papain and/or Alcalase 2.4 L;

In step (2), it is said to keep the ethanol concentration in enzymolysis system at 55% (v/v), which is maintained by outside supplementing ethanol solution;

Said condensation procedure in step (2) is to ensure completely removing ethanol, and the water activity of liquor flavor-enhancing peptide is not higher than 0.3.

The liquor flavor-enhancing peptide produced by the above-mentioned method can be used in flavoring liquor, with addition amount of 50-100 ppm.

In comparison with the current techniques, the present invention has the following advantages and effects:

(1) The present invention uses chloroform/ethanol mixed liquor to elute the alcohol-soluble substances from the defatted soybean meal, which substances are mainly steroids, fats and oligosaccharides. The pre-processing to remove alcohol-soluble substances can raise following enzymolysis efficiency of the defatted soybean meal, avoiding development of bitter taste and abnormal taste in the hydrolysate.

(2) In the present invention, 55% ethanol solution is used to hydrolyze the defatted soybean meal, from one side, that 55% of ethanol solution can precipitate and remove the macromolecule protein or polypeptides in the hydrolysate of the defatted soybean meal, so as to avoid precipitation and layering when added in liquors, which could influence appearance of liquors; from the other side, during hydrolysis, that ethanol can absorb abundant abnormal taste due to strong polarity, then ethanol is easy to be removed during the following condensation procedure, providing guarantee for preparing high quality of flavoring peptide.

(3) Because ethanol of high concentration can inhibit the protease activity to decrease hydrolysis efficiency, normally ethanol of high concentration is rarely used as enzymolysis solvent. In the present invention, proteases with high tolerance to ethanol are mainly used as enzymes, for example, Papain, Alcalase, and so on, meanwhile, by re-preparation of these proteases, hydrolysate abundant with flavor enhancement can be prepared.

(4) For the present invention, the operation procedure is simple, the production cost is low, there has no pollution, and when using the flavor-enhancing peptide in liquors, the prepared liquor is clear, transparent, with harmonious and pure taste.

### PARTICULAR EMBODIMENTS

The following particular embodiments are used to further describe the present invention, but are not used to limit the present invention.

Among the following particular embodiments, the sensory evaluation method of liquor is as follows:

The quantitative description analysis is used to make the sensory evaluation of liquor adding liquor flavor-enhancing peptide. The sensory evaluation analysis is carried out in a sensory evaluation room with temperature of 23° C. ±2° C., and the 10 sensory evaluation members are all from laboratory and are trained by perceptual training (5 male, 5 female, aging from 25-35). The test uses Wuliangye and Maotai as the standard reference material respectively. The sensory evaluation members are trained. Aroma, bitter taste, mellow feel and taste satiation are used as sensory index. Then liquor flavor-enhancing peptide with different concentration is added respectively into Wuliangye and Maotai. Each analyst will sniff and taste each sample (about 5 mL), and after evaluating one sample the analyst will rinse the mouth with purified water and then go on with the next sample. For aroma, bitter taste, mellow feel and taste satiation, 15-dot linear coordinate is used, from 0 (the weakest) to 15 (the strongest). The scores of aroma, bitter taste, mellow feel and taste satiation for Wuliangye and Maotai are all set to be 10, and other samples are scored accordingly comparing with the standard reference material. The results of quantitative description analysis are marked using a radar graph.

### Embodiment 1

A preparation method of liquor flavor-enhancing peptide, comprising the following steps:

(1) 1 piece by weight of defatted soybean meal and 3 pieces by weight of chloroform/ethanol (1:1) mixed liquor are taken and mixed; through a colloid mill, the same is centrifugated for 15 min through a centrifugal machine at 8000×g; the obtained precipitate is collected after washed by water for multiple times; and the final precipitate is the defatted soybean meal removal of the alcohol-soluble components;

(2) the defatted soybean meal prepared in step (1) is taken; the protein concentration of the defatted soybean meal in the mixture is adjusted to 7% (w/w) using 55% (v/v) of ethanol aqueous solution; Papain of 0.7% of the defatted soybean meal mass is added therein; at 50° C., keeping the ethanol concentration in the enzymolysis system at 55% (v/v), the same is made enzymolysis for 12 h, and then inactivated and centrifugated for 15 min at 8000×g; the obtained supernatant is the defatted soybean meal hydrolysate abundant with flavoring peptide;

(3) the flavoring peptide solution is condensed to water activity of 0.3; the same is packaged to obtain liquor flavor-enhancing peptide 1.

The sensory evaluation results of Wuliangye and Maotai with/without adding liquor flavor-enhancing peptide 1 are referred to Table 1.

### Embodiment 2

A preparation method of liquor flavor-enhancing peptide, comprising the following steps:

(1) 1 piece by weight of defatted soybean meal and 5 pieces by weight of chloroform/ethanol (1:3) mixed liquor are taken and mixed; through a colloid mill, the same is centrifugated for 15 min through a centrifugal machine at 8000×g; the obtained precipitate is collected after washed by water for multiple times; and the final precipitate is the defatted soybean meal removal of the alcohol-soluble components;

(2) the defatted soybean meal prepared in step (1) is taken; the protein concentration of the defatted soybean meal in the mixture is adjusted to 9% (w/w) using 55% (v/v) of ethanol aqueous solution; Papain of 0.4% of the defatted soybean meal mass and Alcalase 2.4 L of 0.6% are added therein; at 55 ° C., keeping the ethanol concentration in the enzymolysis system at 55% (v/v), the same is made enzymolysis for 16 h, and then inactivated and centrifugated for 15 min at 8000 ×g; the obtained supernatant is the defatted soybean meal hydrolysate abundant with flavoring peptide;

(3) the flavoring peptide solution is condensed to water activity of 0.29; the same is packaged to obtain liquor flavor-enhancing peptide 2.

The sensory evaluation results of Wuliangye and Maotai with/without adding liquor flavor-enhancing peptide 2 are referred to Table 1.

### Embodiment 3

A preparation method of liquor flavor-enhancing peptide, comprising the following steps:

(1) 1 piece by weight of defatted soybean meal and 4 pieces by weight of chloroform/ethanol (1:2) mixed liquor are taken and mixed; through a colloid mill, the same is centrifugated for 15 min through a centrifugal machine at 8000 ×g; the obtained precipitate is collected after washed by water for multiple times; and the final precipitate is the defatted soybean meal removal of the alcohol-soluble components;

(2) the defatted soybean meal prepared in step (1) is taken; the protein concentration of the defatted soybean meal in the mixture is adjusted to 10% (w/w) using 55% (v/v) of ethanol aqueous solution; Alcalase 2.4 L of 1.2% of the defatted soybean meal mass is added therein; at 55° C., keeping the ethanol concentration in the enzymolysis system at 55% (v/v), the same is made enzymolysis for 18 h, and then inactivated and centrifugated for 15 min at 8000 ×g; the obtained supernatant is the defatted soybean meal hydrolysate abundant with flavoring peptide;

(3) the flavoring peptide solution is condensed to water activity of 0.3; the same is packaged to obtain liquor flavor-enhancing peptid 3.

The sensory evaluation results of Wuliangye and Maotai with/without liquor flavor-enhancing peptide 3 are referred to Table 1.

TABLE 1
sensory evaluation of liquor adding flavor-enhancing peptide

| | | Wuliangye | | | | Maotai | | | |
|---|---|---|---|---|---|---|---|---|---|
| | | 0 | 50 | 100 | 150 | 0 | 50 | 100 | 150 |
| items | | ppm | ppm | ppm | ppm | ppm | ppm | ppm | ppm |
| peptide 1 | aroma | 10 | 11 | 12 | 7 | 10 | 12 | 10 | 8 |
| enhancing | bitter | 10 | 9 | 7 | 6 | 10 | 9 | 8 | 6 |
| liquor | taste | | | | | | | | |
| flavor | mellow | 10 | 11 | 13 | 11 | 10 | 13 | 10 | 8 |
| | feel | | | | | | | | |
| | taste | 10 | 12 | 9 | 8 | 10 | 13 | 12 | 12 |
| | satiation | | | | | | | | |
| peptide 2 | aroma | 10 | 11 | 12 | 7 | 10 | 12 | 11 | 8 |
| enhancing | bitter | 10 | 9 | 8 | 7 | 10 | 8 | 6 | 6 |
| liquor | taste | | | | | | | | |
| flavor | mellow | 10 | 11 | 12 | 10 | 10 | 13 | 11 | 8 |
| | feel | | | | | | | | |
| | taste | 10 | 13 | 11 | 9 | 10 | 12 | 11 | 11 |
| | satiation | | | | | | | | |
| peptide 3 | aroma | 10 | 11 | 12 | 8 | 10 | 12 | 9 | 8 |
| enhancing | bitter | 10 | 9 | 7 | 6 | 10 | 9 | 8 | 6 |
| liquor | taste | | | | | | | | |
| flavor | mellow | 10 | 11 | 13 | 11 | 10 | 13 | 11 | 9 |
| | feel | | | | | | | | |
| | taste | 10 | 11 | 13 | 10 | 10 | 12 | 11 | 10 |
| | satiation | | | | | | | | |

From Table 1, the results showed that the flavor-enhancing peptide prepared by the method of the present invention can obviously improve the liquor flavor (Wuliangye and Maotai). In these two kinds of liquor, by adding the three flavor-enhancing peptides, the bitter taste can be notably decreased, for which the score of bitter taste is becoming lower and lower with increasing of the addition amount; in general, the flavoring function of the three flavor-enhancing peptides for liquor is similar: in Wuliangye, good sensory evaluation score is achieved by adding 50 ppm flavor-enhancing peptide; while in Maotai, better sensory evaluation score is achieved by adding 100 ppm flavor-enhancing peptide. Therefore, liquor flavor is remarkably improved by adding microcontent of liquor flavor-enhancing peptide, indicating the liquor flavor-enhancing peptide prepared by the method of the present invention having a prosperous application future.

The above embodiments are optimal examples of the present invention, but the implementation manners are not limited to the above-mentioned embodiments, and any other revise, modifications, replacement, combination and simplification without departing from the spirit and principle of the present invention are equivalent substitutions, and are included in the protection scope of the present invention.

1. A method for preparing liquor flavor-enhancing peptide, characterized by comprising the following steps:
(1) to remove the alcohol-soluble components from defatted soybean meal: one piece by weight of defatted soybean meal and 3-5 pieces by weight of chloroform/ethanol are mixed uniformly; after treated by colloid mill and centrifuge, the precipitate is collected after washed by water for multiple times; and the final precipitate is the defatted soybean meal removal of the alcohol-soluble components;
(2) to control enzymolysis of the defatted soybean meal: 55% (v/v) of ethanol aqueous solution is added into the defatted soybean meal prepared in step (1) until the protein concentration of the defatted soybean meal reaching to 7%-10% (w/w) of the mixture; then the protease is added therein; the enzymolysis reaction is carried at 50° C.-60° C. for 12-18 h, keeping stable ethanol concentration at (55% (v/v)); after inactivated and centrifugated,the obtained supernatant is the soybean protein hydrolysate riched in flavor peptide; and then the same is condensed to obtain liquor flavor-enhancing peptide; using the mass of defatted soybean meal as calculation reference, the addition amount of protease accounts for 0.5%-1.2%; and said mixture means the system composed of the defatted soybean meal produced in step (1) and ethanol aqueous solution of 55% (v/v);
said condensation procedure in step (2) is to remove completely ethanol, and the water activity of the liquor flavor-enhancing peptide is not higher than 0.3.

2. The preparation method of liquor flavor-enhancing peptide as claimed in claim 1, characterized in that said centrifugation is to centrifugate for 15 min at 8000×g.

3. The preparation method of liquor flavor-enhancing peptide as claimed in claim 1, characterized in that, in said chloroform/ethanol mixed liquor in step (1), the ratio by volume of chloroform to ethanol is 1:(1-3).

4. The preparation method of liquor flavor-enhancing peptide as claimed in claim 1, characterized in that said protease in step (2) is Papain and/or Alcalase2.4 L.

5. A liquor flavor-enhancing peptide, characterized in that it is prepared by the method as claimed in any of claims 1 to 4.

6. Uses of the liquor flavor-enhancing peptide as claimed in claim 5 in liquor flavoring.

7. Uses of the liquor flavor-enhancing peptide in liquor flavoring as claimed in claim 6, characterized in that the addition amount of the liquor flavor-enhancing peptide in liquor is 50-100 ppm.
